(19) United States
(12) Patent Application Publication
(10) Pub. No.: US 2015/0073387 A1
(43) Pub. Date: Mar. 12, 2015

(54) BIFURCATED DRAINAGE TUBING FOR RAPID PARACENTESIS

(71) Applicant: Steven Bushnell, Sacramento, CA (US)

(72) Inventor: Steven Bushnell, Sacramento, CA (US)

(21) Appl. No.: 14/022,801

(22) Filed: Sep. 10, 2013

(51) Int. Cl. A61M 1/00

(52) U.S. Cl. CPC A61M 1/0023
USPC 604/507; 604/319

(57) ABSTRACT

Paracentesis apparatus providing increased fluid transfer capacity, comprising a single centesis needle and at least two vacuum sources. The centesis needle is connected to the vacuum sources by a conduit which is split to form a flow path to each vacuum source. The conduit may comprise flexible tubing, and may include an in-line valve and a connector for engaging the vacuum sources. The vacuum sources may comprise evacuated bottles. Albumin may be administered to patients undergoing paracentesis.

### FIELD OF THE INVENTION

The present invention relates to medical procedures, and more particularly, to method and apparatus for increasing throughput of flow in the course of a paracentesis.

### BACKGROUND OF THE INVENTION

From time to time, it becomes necessary to remove ascites or fluid from the abdominal area of a patient. In most cases, this is to reduce internal pressure, although other reasons may exist. In conventional practice, a needle is inserted through the abdominal wall into the patient and is connected by tubing to a vacuum source such as an evacuated bottle having sufficiently strong partial vacuum as to draw fluid from the abdomen. Where large volumes of fluid must be removed, care must be taken to moderate the rate of removal so as not to cause sudden drops in blood pressure.

In the conventional form of paracentesis the needle is connected by a tube to a single vacuum source or bottle.

Despite the necessity of considering blood pressure, it would be desirable to greatly reduce the time necessary to accomplish paracentesis. For example, using conventional practice, it may require two hours or more to remove ten liters of fluid. Obviously, the rate of fluid extraction could be greatly expedited without threat to stability of blood pressure. It would be undesirable to utilize a second needle and associated apparatus, as this introduces a second and undesirable penetration of body tissues, complication to the medical procedure, and increased costs.

### SUMMARY OF THE INVENTION

The present invention addresses the above stated goal by providing a way to expedite removal of fluid from the abdomen while utilizing only one penetration of the abdominal wall or other bodily tissue. To this end, apparatus to perform a paracentesis operation may utilize a centesis needle connected to a tube, a manifolding fitting which divides flow from the tube into several separate streams, and one additional tube for each of the separate streams. Each of these additional tubes may be connected to an evacuated bottle or other vacuum source.

The novel apparatus and associated method may utilize conventional centesis needle, tubing, fittings, and vacuum sources, and therefore need not require special or dedicated apparatuses.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a plan view of paracentesis apparatus according to at least one aspect of the invention.

FIG. 2 is a block diagram of steps of a method of using the paracentesis apparatus of FIG. 1.

### DETAILED DESCRIPTION

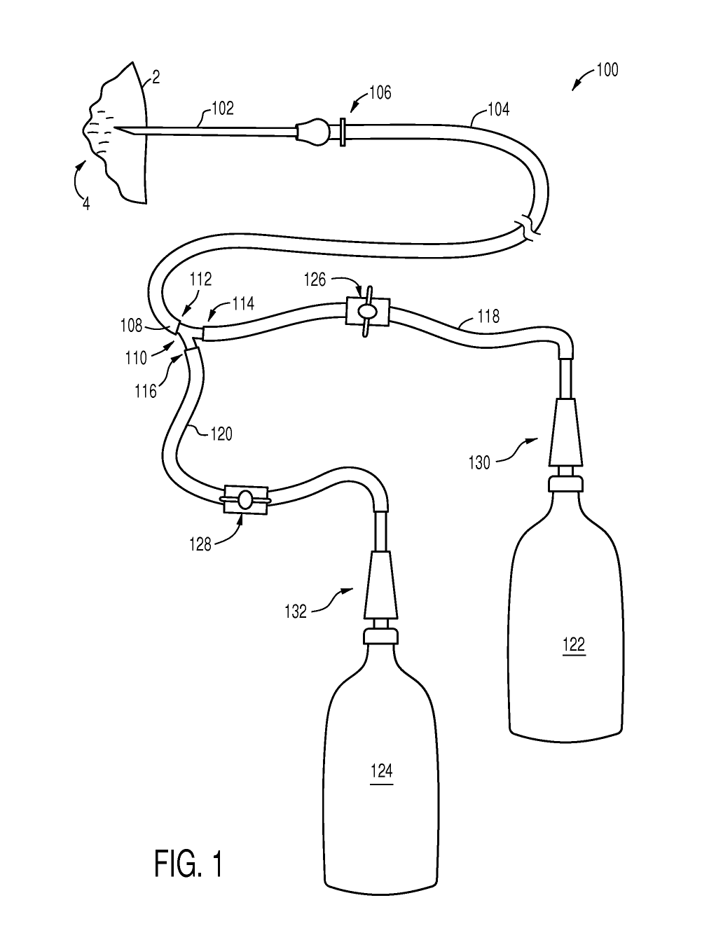

Referring first to FIG. 1, according to at least one aspect of the invention, there is shown paracentesis apparatus 100 including a centesis needle 102 which has been installed in a mammalian body 2 containing ascites 4 such as through the abdominal wall (not explicitly shown) for purposes of extraction of the ascites 4 from the mammalian body 2. Portions of the centesis needle 2 project to the exterior of the mammalian body 2.

The paracentesis apparatus 100 may comprise a first conduit 104 having a proximal end 106 disposed in fluid communication with the centesis needle 102, and a distal end 108. The first conduit 104 divides or splits into two flow paths. The point of split or bifurcation may occur for example at a manifolding fitting 110 which comprises a first port 112 which is compatible with the first conduit 104, and at least two additional ports 114, 116. The manifolding fitting 110 may be described as a three-way fitting comprising the first port 112 and the two additional ports 114, 116.

Each of the two flow paths is contained within one of two additional conduits 118 or 120. A number of vacuum sources such as evacuated bottles 122, 124 is connected to the additional conduits 118, 120. The terms vacuum source and evacuated bottle will generally be used interchangeably hereinafter, as feasibility and context dictate. The number of vacuum sources is equal to the number of the additional ports 114, 116 and to the number of additional conduits 118, 120. Just as the first conduit 114 is disposed in fluid communication with the centesis needle 102 and the first port 112, the additional conduits 118, 120 are disposed in fluid communication with respective additional ports 114, 116, and with the vacuum sources. In summary, flow from the centesis needle 102 splits into alternative flow paths represented by the additional conduits 118, 120, and ultimately to the vacuum sources.

The first conduit 104 and the additional conduits 118, 120 may be provided for example by flexible tubing. It should also be noted that description of the paracentesis apparatus 100 in terms of single conduits such as the first conduit 104 and the additional conduits 118 and 120 need not imply that these conduits each consist of only one continuous section of tubing. It would be possible to provide plural sections of tubing or other conduits joined by suitable connectors (this option not shown), provided that the divided flow scheme described above be present. Also, it would be possible to increase the number of vacuum sources beyond the two explicitly shown.

Flow to each of the evacuated bottles 122 or 124 may be controlled by a valve arrangement comprising valves 126, 128 each disposed serially in one of the additional conduits 118, 124 respectively. Each valve 126, 128 enables discontinuing communication of its associated evacuated bottle 122 or 124, thereby selectively discharging ascites into only one of the evacuated bottles 122 and 124, or in some implementations of the invention, modifying flow rate in each of the additional conduits 118, 120.

Of course, it would be possible to provide only one of the additional conduits 118 or 124 with a valve 126, 128 if desired. However, greatest versatility of the system is enabled when a valve 126 or 128 is disposed serially in each one of the additional conduits 118, 120.

Each one of the additional conduits 118, 120 preferably further comprises a respective connector 130, 132 which is configured to engage the vacuum source and to maintain its respective additional conduit in fluid communication with the vacuum source. Each of the connectors 130, 132 may comprise spiked tubing.

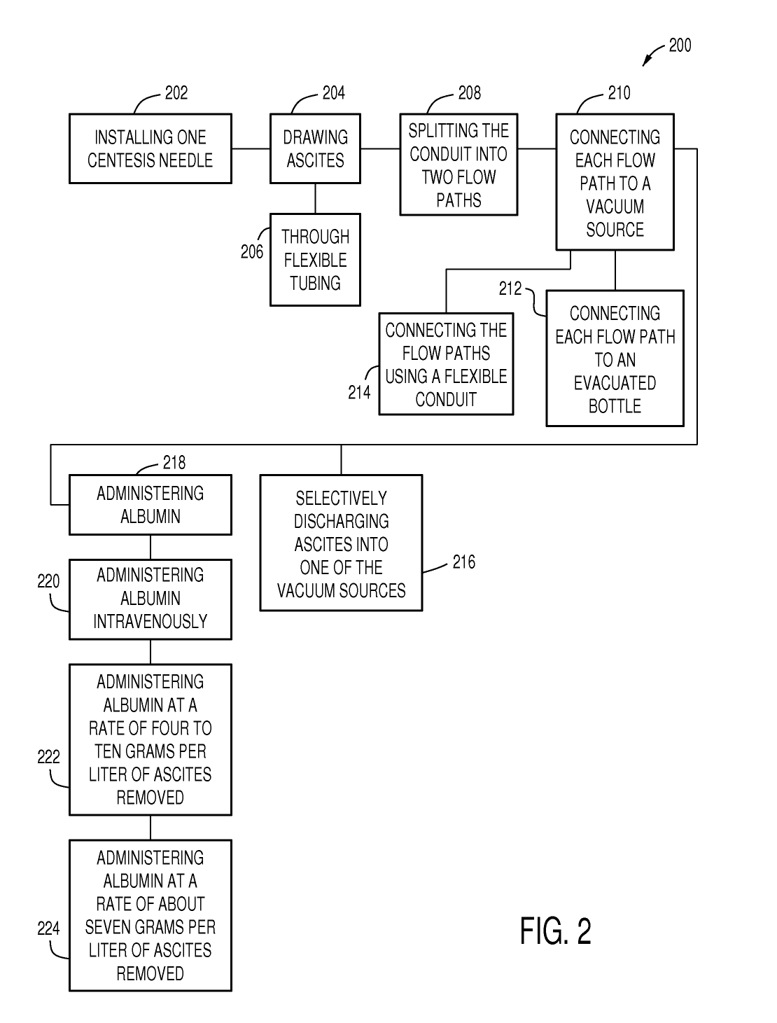

In use, the apparatus 100 may be utilized to perform a method 200 of conducting extraction of ascites from a mammalian body, as shown in FIG. 1. As summarized in FIG. 2, the method 200 may comprise the following steps. In a step 202, one and only one centesis needle such as the centesis needle 102 is installed in the mammalian body so as to be in fluid communication with the ascites, and to project to the exterior of the mammalian body. In a step 204, ascites is drawn from the centesis needle through a conduit such as the first conduit 104. The step 204 may comprise a further step 206 of drawing ascites from the centesis needle through flexible tubing. The method 200 may include a step 208 of splitting the conduit, such as the first conduit 104, into at least two flow paths. The method 200 may include a step 210 of connecting each one of the flow paths to a dedicated vacuum source. As employed herein, the term dedicated signifies that the dedicated element is associated with a respective one of the additional conduits 118 or 120. The step 210 may comprise a further step 212 of connecting each one of the flow paths to one evacuated bottle such as the evacuated bottles 122, 124.

The step 210 may comprise a further step 214 of connecting each one of the flow paths to one evacuated bottle using a flexible conduit for at least one and preferably both of the two flow paths.

The method 200 may comprise a step 216 of selectively discharging ascites into one of the vacuum sources. This may be done using the valves 126, 128.

In a step 218, the method 200 may comprise administering albumin to patients undergoing paracentesis. The step 218 may comprise a further step 220 of administering albumin to the patient intravenously. The step 218 may comprise a further step 222 of administering albumin at a rate of four grams to ten grams of albumin for each liter of ascites removed. In a most preferred implementation, the step 222 may comprise a further step 224 of administering albumin at the rate of about seven grams of albumin for each liter of ascites removed. It is preferred to use about a twenty-five percent solution of albumin when administering albumin at the rate of seven grams of albumin per liter of ascites removed.

It is not necessary to penetrate the abdominal wall to gain access to fluid being drained, although such practice is conventional. Depending upon the particular medical procedure, the same apparatus and method could be utilized while penetrating other body tissues, such as a lateral or dorsal surface, or by utilizing a natural body opening such as a rectal opening, vaginal opening, urethral opening, or exploiting a pre-existing wound or incision.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. Paracentesis apparatus comprising:
a centesis needle;
a first conduit having a proximal end disposed in fluid communication with the centesis needle, and a distal end;
a manifolding fitting comprising a first port which is compatible with the first conduit, and at least two additional ports, wherein the manifolding fitting divides flow from the first conduit into at least two fluid paths; and
a number of vacuum sources equal to the number of the additional ports; and
a number of additional conduits equal to the number of additional ports, wherein each additional conduit is disposed in fluid communication with one of the additional ports of the manifolding fitting and with one of the vacuum sources.

2. The paracentesis apparatus of claim 1, wherein the first conduit comprises flexible tubing.

3. The paracentesis apparatus of claim 1, wherein each one of the additional conduits comprises flexible tubing.

4. The paracentesis apparatus of claim 1, wherein
the manifolding fitting is a three-way fitting comprising a first port and two additional ports,
the additional conduits comprise two flexible conduits; and
the number of vacuum sources is two.

5. The paracentesis apparatus of claim 1, further comprising a valve disposed serially in at least one of the additional conduits.

6. The paracentesis apparatus of claim 1, further comprising a valve disposed serially in each one of the additional conduits.

7. The paracentesis apparatus of claim 1, wherein each one of the additional conduits further comprises a connector which is configured to engage the vacuum source and to maintain its respective additional conduit in fluid communication with the vacuum source.

8. The paracentesis apparatus of claim 7, wherein the connector comprises spiked tubing.

9. A method of conducting extraction of ascites from a mammalian body, comprising:
installing one and only one centesis needle in the mammalian body so as to be in fluid communication with the ascites, and to project to the exterior of the mammalian body;
drawing ascites from the centesis needle through a conduit;
splitting the conduit into at least two flow paths; and
connecting each one of the flow paths to a dedicated vacuum source.

10. The method of claim 9, wherein drawing ascites from the centesis needle through a conduit comprises drawing ascites from the centesis needle through flexible tubing.

11. The method of claim 9, further comprising selectively discharging ascites into one of the vacuum sources.

12. The method of claim 9, wherein connecting each one of the flow paths to a dedicated vacuum source comprises connecting each one of the flow paths to one evacuated bottle.

13. The method of claim 12, wherein connecting each one of the flow paths to one evacuated bottle comprises connecting each one of the flow paths to one evacuated bottle using a flexible conduit for at least one of the flow paths.

14. The method of claim 9, further comprising administering albumin to the patient.

15. The method of claim 14, wherein administering albumin to the patient comprises administering albumin to the patient intravenously.

16. The method of claim 14, wherein administering albumin to the patient comprises administering albumin at a rate of four grams to ten grams of albumin for each liter of ascites removed.

17. The method of claim 14, wherein administering albumin to the patient comprises administering albumin at the rate of about seven grams of albumin for each liter of ascites removed.
